(19) United States
(12) Patent Application Publication
(10) Pub. No.: US 2015/0209536 A1
(43) Pub. Date: Jul. 30, 2015

(54) ENDOTRACHEAL TUBE WITH DUAL PORT SUBGLOTTIC SECRETION SUCTIONING

(71) Applicant: TELEFLEX MEDICAL INCORPORATED, Research Triangle Park, NC (US)

(72) Inventor: Gary James ROTH, Wake Forest, NC (US)

(21) Appl. No.: 14/681,634

(22) Filed: Apr. 8, 2015

Related U.S. Application Data
(63) Continuation of application No. 13/292,730, filed on Nov. 9, 2011, now abandoned.

(51) Int. Cl. A61M 16/04

(52) U.S. Cl. CPC A61M 16/0463; A61M 16/0434

(57) ABSTRACT

An endotracheal tube assembly comprises an elongate tube having an outer wall defining an outer surface, the tube defining an elongate central lumen and an elongate suction lumen, and an inflatable cuff attached on a distal end portion of the elongate tube. The elongate tube defines a first suction opening having a normal vector parallel to a tangent to an outer circumference of a transverse cross-section of the outer wall of the tube next to said first suction opening, the first suction opening being in fluid communication with the suction lumen.

### CROSS-REFERENCE TO RELATED APPLICATION

This application is a continuation of and claims priority to U.S. patent application Ser. No. 13/292,730, filed Nov. 9, 2011, the disclosure of which is incorporated by reference in its entirety.

### FIELD OF THE INVENTION

The present invention generally relates to an endotracheal tube. More particularly, the present invention pertains to an endotracheal tube with subglottic secretion suctioning.

### BACKGROUND OF THE INVENTION

Endotracheal tubes are commonly used for airway management, mechanical ventilation, and as a drug delivery device when intravenous delivery cannot be established. An endotracheal tube is inserted through a process called intubation. During intubation, the endotracheal tube is inserted into the trachea through the patient's mouth, in order to keep the airway open and to allow air to reach the lungs. Endotracheal tubes often include a cuff to prevent air leaks during mechanical ventilation. This cuff is most commonly positioned at the distal end of the tube and can be inflatable.

Additionally, it is possible for undesirable subglottic secretions to collect around the top of the cuff. In order to prevent ventilator-associated pneumonia (VAP), it can be beneficial to remove these secretions by some means. One way to remove these secretions is to remove the endotracheal tube and then re-intubate the patient. However, continued re-intubation can be traumatic to the patient's trachea and is therefore not preferable. Alternatively, suction can be applied to remove these secretions through an additional lumen in the ET tube post intubation.

It is therefore desirable to provide a device and method for suctioning these subglottic secretions from the tracheal mucosa without having to remove the endotracheal tube.

### SUMMARY OF THE INVENTION

The foregoing needs are met, to a great extent, by the present invention, wherein in some embodiments an endotracheal tube that is capable of overcoming the disadvantages described herein at least to some extent is provided.

In accordance with an embodiment of the present invention, an endotracheal tube assembly includes an elongate tube having an outer wall defining an outer surface. The endotracheal tube defines an elongate central lumen and an elongate suction lumen. An inflatable cuff is attached on a distal end portion of the elongate tube. The outer wall of the elongate tube defines an opening for the suction lumen proximate to a proximal end of the inflatable cuff. Additionally, a diffuser cap is positioned on a distal portion of the outer wall of the tube over the distal opening for the suction lumen to define a first suction opening. The diffuser cap is also configured to include normal vector parallel to a tangent to an outer circumference of a transverse cross-section of the outer wall of the tube next to said first suction opening.

In accordance with another embodiment of the present invention, the first suction opening defined by the diffuser cap can be in fluid communication with the opening for the suction lumen and can also be in fluid communication with the suction lumen. Additionally, the diffuser cap can define a second suction opening having a normal vector parallel to another tangent to the outer circumference of the transverse cross-section of the outer wall of the tube next to said second suction opening. The first suction opening can be oriented facing in a direction tangent to a first edge portion of the outer surface of the outer wall of the tube, and the second suction opening can be oriented facing in a direction tangent to a second edge portion of the outer surface of the outer wall of the tube. The diffuser cap can also extend around a partial circumference of the distal end portion of the elongate tube to define an annular flow channel. The first and second suction openings can be disposed on opposite ends of said channel each in fluid communication with the opening of the suction lumen.

In accordance with still another embodiment of the present invention, the diffuser cap defines a third suction opening facing the proximal end of the cuff, the third suction opening being in fluid communication with the annular channel between the first and second suction openings. The diffuser cap can be formed from one of a plastic or a resin and can be formed integrally with the endotracheal tube. Alternately, the diffuser cap can be formed separately and bonded to the endotracheal tube with heat, glue, or another suitable means.

In accordance with another embodiment of the present invention, an endotracheal tube includes an elongate tube having an outer wall defining an outer surface, the tube defining an elongate central lumen and an elongate suction lumen. An inflatable cuff is attached on a distal end portion of the elongate tube. Additionally, the elongate tube defines a first suction opening having a normal vector parallel to a tangent to an outer circumference of a transverse cross-section of the outer wall of the tube next to said first suction opening. Additionally, the first suction opening is in fluid communication with the suction lumen.

In accordance with another embodiment of the present invention, the elongate tube can define a second suction opening having a normal vector parallel to another tangent to an outer circumference of the transverse cross-section of the outer wall of the tube next to said second suction opening. The first suction opening can be oriented to face in a direction tangent to a first edge portion of the outer surface of the outer wall of the tube. The second suction opening can be oriented to face in a direction tangent to a second edge portion of the outer surface of the outer wall of the tube. The first edge portion and second edge portion can be separated by a partial circumference of the distal end portion of the elongate tube, which defines an annular flow channel through which the first and second suction openings are disposed on opposite ends of said channel each in fluid communication with the suction lumen. A diffuser cap can be disposed over an outer surface of a distal end portion of the elongate tube to define the annular flow channel and first and second suction openings. The elongate tube defines a third suction opening facing the proximal end of the cuff.

In accordance with still another embodiment of the present invention, an elongate tube includes an outer wall defining a generally cylindrical elongate central lumen and a generally cylindrical outer surface and also defining an elongate suction lumen. An inflatable cuff is attached near a distal end of the elongate tube, and the inflatable cuff has a proximal end and a distal end. The outer wall of the elongate tube defines an opening for the suction lumen proximal to the proximal end of the inflatable cuff. Additionally, a diffuser bridge is positioned on a portion of the outer wall of the tube. The diffuser bridge is raised over the opening to the suction lumen such that it covers the opening for the suction lumen. A wall of the diffuser bridge and the tube together defines two suction openings each facing parallel to respective lines tangent to respective portions of an outer circumference of the tube.

In accordance with yet another embodiment of the present invention, the wall of the diffuser bridge can define a third opening facing the proximal end of the inflatable cuff in its inflated state. The diffuser bridge can be formed a plastic or a resin, or any other suitable material. The diffuser bridge can be formed integrally with the endotracheal tube or can be bonded to the endotracheal tube.

In accordance with even another embodiment of the present invention, an endotracheal tube assembly includes an elongate tube having an outer wall defining a generally cylindrical elongate central lumen and a generally cylindrical outer surface. The elongate tube defines an elongate suction lumen. The elongate tube also has a longitudinal axis extending along the length of the tube and a transverse axis extending across the width of the tube. The assembly also includes an inflatable cuff attached near a distal end of the elongate tube, wherein the inflatable cuff has a proximal end and a distal end. The outer wall of the elongate tube also defines an opening for the suction lumen proximal to the proximal end of the inflatable cuff. Additionally, the device includes a diffuser ring positioned around the outer wall of the elongate tube such that the diffuser ring at least partially covers the opening for the suction lumen. The diffuser ring includes a first diffuser ring channel parallel to the longitudinal axis of the elongate tube and a second diffuser ring channel parallel to the transverse axis of the elongate tube.

In accordance with even another embodiment of the present invention, the endotracheal tube can include the diffuser ring being formed from one of a plastic or a resin. The endotracheal tube can be formed integrally with the endotracheal tube, or alternately, the diffuser ring being bonded to the endotracheal tube. The diffuser ring can be bonded to the endotracheal tube with one of heat treatment or an adhesive.

In accordance with still another embodiment of the present invention, a top and a bottom edge of the diffuser ring can be beveled. Additionally, edges of the first and second diffuser ring openings can be beveled. A point where the first diffuser ring opening intersects the second diffuser ring opening can be positioned to be over the opening to the suction lumen. The diffuser ring can also have a first thickness adjacent to the opening to the suction lumen and a second thickness opposite the opening to the suction lumen and wherein the first thickness is greater than the second thickness.

In accordance with another embodiment of the present invention an endotracheal tube assembly includes an elongate tube having an outer wall defining a generally cylindrical elongate central lumen and also defining an elongate suction lumen. The endotracheal tube also includes an inflatable cuff attached near a distal end of the elongate tube. The inflatable cuff has a proximal end, a distal end, an inner surface, and an outer surface. The outer wall of the elongate tube defines an opening for the suction lumen proximal to the proximal end of the inflatable cuff. A diffuser cap is positioned over the opening for the suction lumen. The diffuser cap also defines an opening tangent to an outer surface of the outer wall of the endotracheal tube.

In accordance with another embodiment of the present invention the opening defined by the diffuser cap can be in fluid communication with the opening for the suction lumen and is in fluid communication with the suction lumen. The diffuser cap can also define two openings. A first opening can be tangent to a leftmost edge of the outer surface of the outer wall of the endotracheal tube, and a second opening can be tangent to a rightmost edge of the outer surface of the outer wall of the endotracheal tube. Additionally, the diffuser cap can define a distal opening oriented perpendicular to an outer surface of the outer wall of the endotracheal tube, such that the distal opening is parallel to a proximal surface of the inflatable cuff when it is in its inflated state. The diffuser cap can be formed from a plastic or a resin, and can be formed integrally with the endotracheal tube or bonded to the endotracheal tube. If the diffuser cap is bonded to the endotracheal tube it can be done with heat or adhesive.

In accordance with another embodiment of the present invention an endotracheal tube assembly includes an elongate tube having an outer wall defining a generally cylindrical elongate central lumen and also defining an elongate suction lumen. An inflatable cuff is attached near a distal end of the elongate tube, wherein the inflatable cuff has a proximal end and a distal end and an inner surface and an outer surface. The outer wall of the elongate tube defines an opening for the suction lumen proximal to the proximal end of the inflatable cuff the opening for the suction lumen having an outer circumference. A diffuser bridge is raised above the surface of the opening to the suction lumen. The diffuser bridge defines two openings. A first opening is parallel to a line tangent to a left side of the outer circumference of the opening for the suction lumen and a second opening is parallel to a line tangent to a right side of the outer circumference of the opening for the suction lumen.

In accordance with another embodiment of the present invention, the opening defined by the diffuser cap can be in fluid communication with the opening for the suction lumen and is in fluid communication with the suction lumen. The diffuser cap can be formed from a plastic or a resin, and can be formed integrally with the endotracheal tube or bonded to the endotracheal tube. If the diffuser cap is bonded to the endotracheal tube, it can be done with heat or adhesive.

In accordance with still another aspect of the present invention an endotracheal tube assembly includes an elongate tube having an outer wall defining a generally cylindrical elongate central lumen and a generally cylindrical outer surface and also defining an elongate suction lumen. An inflatable cuff is attached near a distal end of the elongate tube, wherein the inflatable cuff has a proximal end and a distal end. Also, the outer wall of the elongate tube defines an opening for the suction lumen proximal to the proximal end of the inflatable cuff the opening for the suction lumen having an outer circumference. A diffuser bridge is raised above the surface of the opening. A wall of the diffuser bridge defines two openings wherein a first opening is parallel to a line tangent to a left side of the outer circumference of the opening for the suction lumen and a second opening is parallel to a line tangent to a right side of the outer circumference of the opening for the suction lumen. The wall of the diffuser bridge defines a third opening positioned parallel to a surface of the proximal end of the inflatable cuff in its inflated state.

In accordance with another embodiment of the present invention, the opening defined by the diffuser cap can be in fluid communication with the opening for the suction lumen and is in fluid communication with the suction lumen. The diffuser cap can be formed from a plastic or a resin, and can be formed integrally with the endotracheal tube or bonded to the endotracheal tube. If the diffuser cap is bonded to the endotracheal tube it can be done with heat or adhesive.

There has thus been outlined, rather broadly, certain embodiments of the invention in order that the detailed description thereof, herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates a side view of an endotracheal tube in accordance with an embodiment of the invention.

FIG. 2 illustrates a sectional view along axis “B” of the endotracheal tube shown in FIG. 1.

FIG. 3 illustrates a partially sectional view of the endotracheal tube with dual port subglottic secretion suctioning in accordance with an embodiment of the invention.

FIG. 4 illustrates a view of the endotracheal tube with dual port subglottic secretion suctioning in accordance with an embodiment of the invention.

FIG. 5 illustrates a reverse view of the endotracheal tube with dual port subglottic secretion suctioning illustrated in FIG. 4 in accordance with an embodiment of the invention.

FIG. 5A illustrates a sectional view taken along the transverse axis “T” shown in FIG. 5.

FIG. 6 illustrates a partially sectional view of the endotracheal tube with dual port subglottic secretion suctioning in accordance with an embodiment of the invention.

FIG. 7 illustrates a view of a diffuser cap for coupling to an endotracheal tube to provide dual port subglottic secretion suctioning in accordance with an embodiment of the invention.

FIG. 8 illustrates a partial view of an endotracheal tube with multi-port subglottic secretion suctioning in accordance with another embodiment of the present invention.

FIG. 9 illustrates a partial view of an endotracheal tube with multi-port subglottic secretion suctioning in accordance with an embodiment of the invention.

FIG. 10 illustrates a partial side view of the endotracheal tube with multi-port subglottic secretion suctioning illustrated in FIG. 9 in accordance with an embodiment of the invention.

FIG. 11 illustrates a sectional view of the endotracheal tube with multi-port subglottic secretion suctioning illustrated in FIGS. 9 and 10 in accordance with an embodiment of the invention.

### DETAILED DESCRIPTION

The present invention provides in some embodiments, an endotracheal tube with dual port subglottic secretion suction. The endotracheal tube is an elongate tube having an outer wall defining an elongate central lumen and an elongate suction lumen. An inflatable cuff is attached near a distal end of the elongate tube. The suction lumen terminates in a suction opening near a distal end of the endotracheal tube. The wall of the endotracheal tube defines the suction opening, such that it includes a dual port configuration.

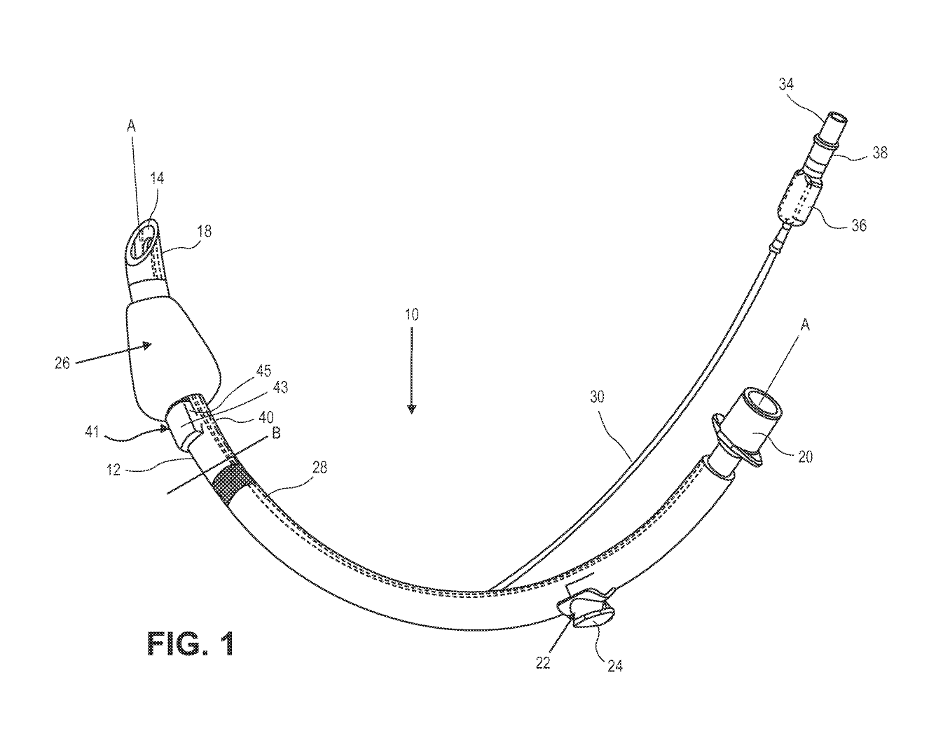

The invention will now be described with reference to the drawing figures, in which like reference numerals refer to like parts throughout. FIG. 1 illustrates an endotracheal tube 10 in accordance with the invention. The endotracheal tube 10 includes an outer wall 12 which defines a generally cylindrical central lumen 14. The outer wall 12 also defines an elongate suction lumen 16. The endotracheal tube 10 has a longitudinal axis “A” extending through the length of the tube 10 and one or more transverse axis “B” extending across the tube 10. The endotracheal tube has a distal end 18, which is inserted into the patient's trachea during the intubation procedure, and a proximal end 20, which is coupled to the respirator or other source of air for the patient. The endotracheal tube 10 can also include a connector 22 mounted on the outer wall 12 of the endotracheal tube. As shown in FIG. 1, the connector can also include a cover 24 shown in a closed position in FIG. 1.

FIG. 1 also illustrates an inflation cuff 26 positioned proximate to the distal end 18 of the endotracheal tube 10. The inflation cuff 26 can be inflated via an inflation lumen 28 defined by the outer wall 12 of the endotracheal tube 10. As shown in FIG. 1, an inflation tube 30 extends from the inflation lumen 28. A distal end of the inflation tube 30 is inserted into the inflation lumen 28 and a proximal end 34 can be coupled to a source of air for inflation. A pilot 36 and a valve 38 can be coupled to the proximal end 34 of the inflation tube in order to keep air in the cuff 26, once it is inflated. The endotracheal tube 10 can also include an x-ray opaque stripe 40 extending through the outer wall 12 of the endotracheal tube 10, so that proper placement of the tube 10 can be confirmed via x-ray, if necessary.

Also, as illustrated in FIG. 1, a diffuser cap 41 is positioned over a first suction opening (not shown) defined by the outer wall 12 of the endotracheal tube 10 and positioned near a distal end 18 of the endotracheal tube 10. The first suction opening is in fluid communication with the suction lumen 16. The diffuser cap 41 includes a diffuser cap wall 43 which defines at least one diffuser cap opening 45, which is in fluid communication with the first suction opening and the suction lumen 16. In one embodiment of the invention, diffuser cap opening 45 can have a normal vector parallel to a tangent to an outer circumference of a transverse cross-section along axis “B” of the outer wall 12 of the endotracheal tube 10 next to the first suction opening.

Alternately, the endotracheal tube 10 can define the first suction opening such that it has a normal vector parallel to a tangent to the outer circumference of the transverse cross-section along axis “B” of the outer wall 12 of the endotracheal tube 10. The first suction opening can also be in fluid communication with the suction lumen. In another embodiment, the outer wall 12 of the endotracheal tube 10 defines a diffuser bridge on a portion of the outer wall 12 the diffuser bridge being raised over and covering the first suction opening. The diffuser bridge and the endotracheal tube 10 can together define a second suction opening facing parallel to a line tangent to an outer circumference of the endotracheal tube 10.

The diffuser cap 41 can be configured to be raised slightly above the surface of the outer wall 12. However, the diffuser cap 41 can also take any configuration suitable for providing fluid communication with the opening to the suction lumen 16 and the suction lumen 16. The diffuser cap 41 can be formed from a plastic such as polyvinyl chloride (PVC) or another resin that can be securely bonded to the endotracheal tube 10. The plastic or resin used to form the diffuser cap 41 can also be of a similar durometer to the material used to form the endotracheal tube 10. The diffuser cap 41 can be bonded to the endotracheal tube 10 using an adhesive, heat bonding, or other suitable method. Alternately, the diffuser cap 41 can be formed integrally with the endotracheal tube 10, and could, therefore be formed from the same material as the endotracheal tube 10. The diffuser cap 41 can also be coupled with or formed using any other method known to one of skill in the art.

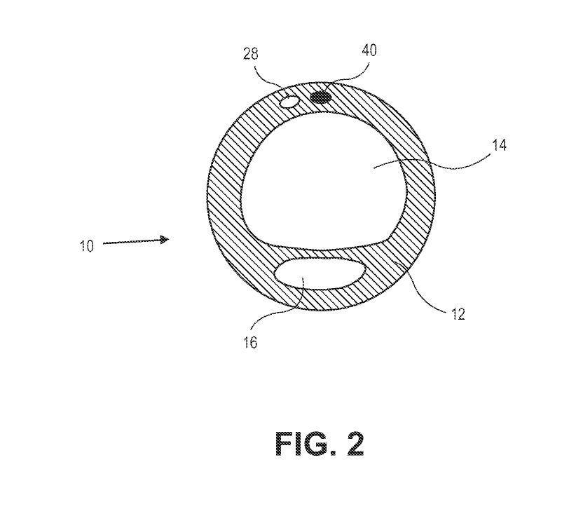

FIG. 2 illustrates a sectional view of an endotracheal tube taken along axis “B” illustrated in FIG. 1. As illustrated in FIG. 2, the endotracheal tube 10 includes a central lumen 14, a suction lumen 16, and an inflation lumen 28 defined by the outer wall 12 of the endotracheal tube 10. The x-ray opaque stripe 40 also extends through the outer wall 12 of the tube 10. The central lumen 14 is shown as generally circular in cross-sectional shape, while the suction lumen 16 is shown as generally elliptical in cross-sectional shape. However, the central lumen 14 and the suction lumen 16 can take any shape which allows for the passage of air or secretions. Additionally, the endotracheal tube 10 can be formed from polyvinyl chloride or any other suitable material for forming an endotracheal tube.

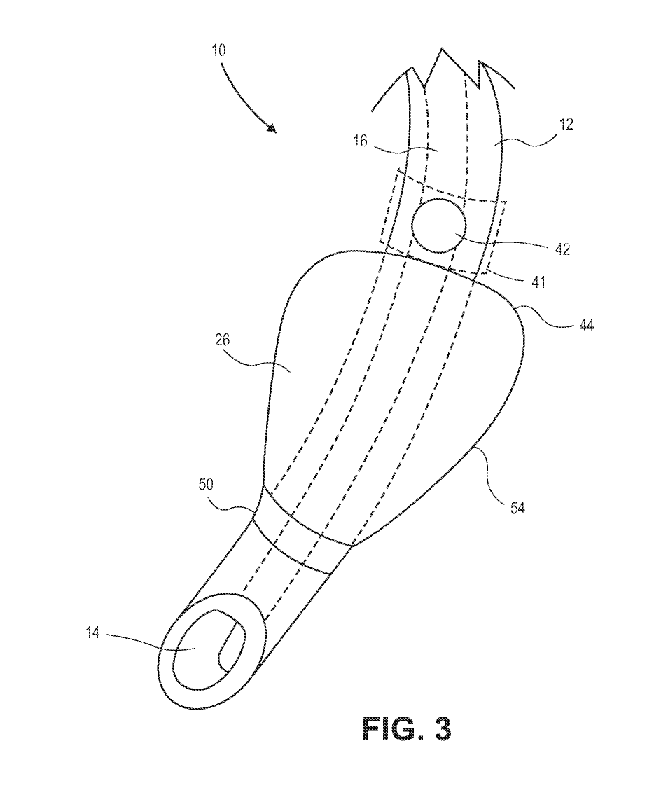

FIG. 3 illustrates a partially sectional view of the endotracheal tube 10 with dual port subglottic secretion suctioning. As illustrated in FIG. 3, the outer wall 12 of the endotracheal tube 10 defines an opening 42 in the suction lumen 16, just proximal of a proximal end 44 of the inflation cuff 26. The opening 42 is disposed underneath the diffuser cap 41, which has been dashed in order to show the opening 42. The opening 42 is in fluid communication with the suction lumen 16.

FIG. 3 also illustrates the inflation cuff 26. The cuff can include a distal end 50 and a proximal end 44. The distal end 50 of the cuff 26 is bonded to the endotracheal tube 10 near its distal end 18. Preferably, the proximal end 44 of the cuff 26 can be folded in, or inverted, such that the outer surface 54 is bonded to the endotracheal tube 10. The bond can be formed by a suitable adhesive, melting, or any other form of bond that will connect the cuff 26 to the tube 10. Inverting the proximal end 44 of the cuff 26, but not the distal end 50, allows for the cuff 26 to increase in diameter from its distal end 50 to its proximal end 44.

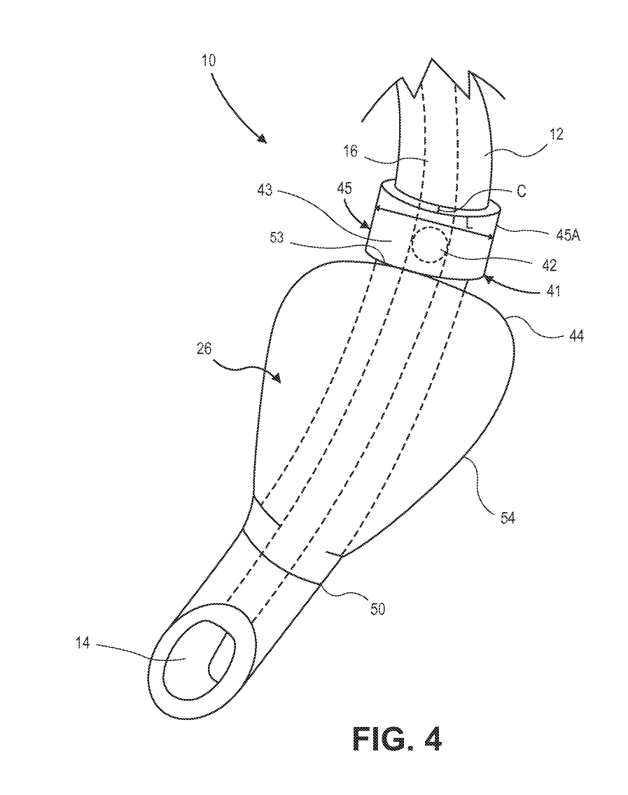

FIG. 4 illustrates a view of the endotracheal tube 10 with dual port subglottic secretion suctioning. As illustrated in FIG. 4, the diffuser cap 41 is disposed just proximal to a proximal end 44 of the inflation cuff 26. The diffuser cap 41 is raised above the surface of the outer wall 12 of the endotracheal tube 10, by a distance “C.” The inflation cuff 26 is shown in its inflated state. The proximal end 44 of the inflation cuff 26 terminates just below the distal edge 53 of the diffuser cap 41.

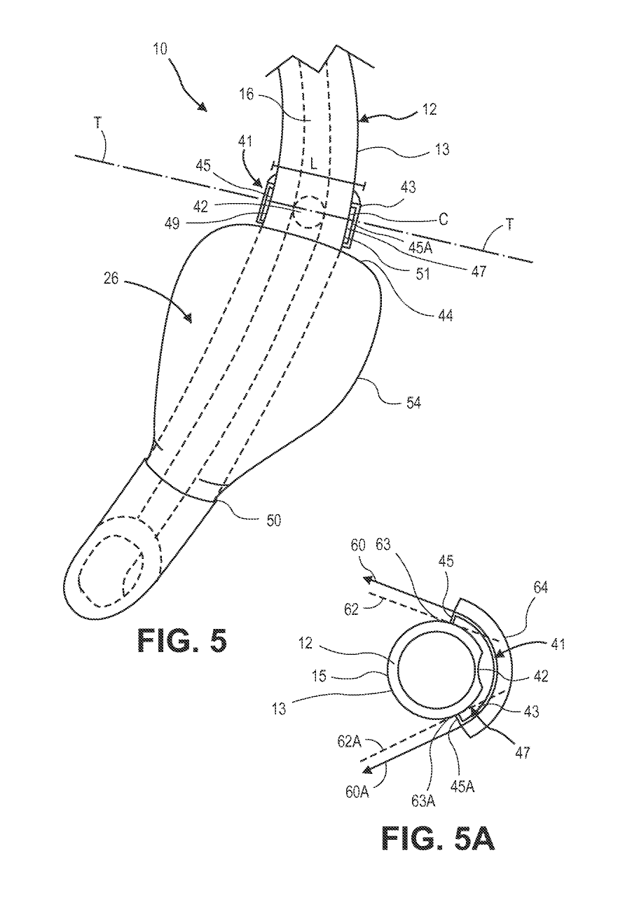

FIG. 5 illustrates a view of the opposite side of the endotracheal tube 10 from that shown in FIG. 4. As illustrated in FIG. 5, the diffuser cap 41 includes two diffuser cap openings 45, 45a defined by the outer wall 43 of the diffuser cap 41. The diffuser cap openings 45, 45a are in fluid communication with the suction lumen opening 42 and the suction lumen 16, thus, creating a flow path for suctioned secretions into the suction lumen 16 and out of the endotracheal tube 10. As shown in FIG. 5, the first diffuser cap opening 45 can have a normal vector parallel to a line tangent to a left side 49 of the outer circumference of the opening 42 for the suction lumen 16; and the second diffuser cap opening 45a can have a normal vector parallel to a line tangent to a right side 51 of the outer circumference of the opening 42 for the suction lumen 16.

In an embodiment of the diffuser cap 41 shown in FIG. 5, the diffuser cap 41 can be positioned on a distal portion 18 of the endotracheal tube 10. The diffuser cap 41 can have the first diffuser cap opening 45 having a normal vector parallel to a tangent to the outer circumference of a transverse cross section of the outer wall 12 of the endotracheal tube 10. The second diffuser cap opening 45a can also have a normal vector parallel to a tangent to the outer circumference of the transverse cross section along axis “B” of the outer wall 12 of the endotracheal tube 10. The first diffuser cap opening 45 can be oriented to face a direction that is tangent to a first edge portion of the outer wall 12 of the endotracheal tube 10.

The endotracheal tube 10 can also define the diffuser cap 41. The endotracheal tube 10 can define the first diffuser cap opening 45, such that it has a normal vector parallel to the tangent to the outer wall 12 of the endotracheal tube 10. The first diffuser cap opening 45 can be in fluid communication with the suction lumen 16. The endotracheal tube 10 can also define the second diffuser cap opening 45a, such that it has a normal vector parallel to the tangent to the outer wall 12 of the endotracheal tube 10. The second diffuser cap opening 45a can be in fluid communication with the suction lumen 16. The first and second diffuser cap openings 45 and 45a can also be oriented to be facing in a direction tangent to an edge portion of the outer wall 12 of the endotracheal tube 10. Alternately, the first and second diffuser cap openings 45 and 45a can face parallel to respective lines tangent to respective portions of an outer circumference of the endotracheal tube 10. The distal end 18 of the endotracheal tube 10 can also define a partial circumference separating the first and second diffuser cap openings 45 and 45a. The partial circumference defines an annular flow channel 47. The first and second diffuser cap openings 45 and 45a are disposed on either end of this annular flow channel 47. The first and second diffuser cap openings 45 and 45a are in fluid communication with the suction lumen 16, via the annular flow channel 47.

Also, as illustrated in FIG. 5, the diffuser cap 41 can be disposed on top of the suction lumen opening 42. The diffuser cap 41 can be raised above the outer surface 13 of the outer wall 12 of the endotracheal tube 10, by a distance of “C.” Distance “C” creates the annular flow channel 47 between the outer wall 43 of the diffuser cap 41 and the outer wall 12 of the endotracheal tube 10. Secretions and mucous can be suctioned into the flow channel 47 and into the suction lumen 16 through suction lumen opening 42. Additionally, the diffuser cap 41 can extend for a circumferential length “L,” which, as illustrated in FIG. 5, extends around a half of the generally cylindrical shape of the endotracheal tube 10. The diffuser cap 41 can extend around a partial circumference of the distal end portion 18. Alternately, the diffuser cap 41 can extend for any length that is suitable for providing multi-port secretion suctioning for the endotracheal tube.

FIG. 5A illustrates a sectional view of the device illustrated in FIG. 5 taken along axis “T”, as illustrated in FIG. 5. In the embodiment of the diffuser cap 41 shown in FIG. 5A, the diffuser cap 41 can extend around a partial circumference of the distal end portion of the elongate tube to define an annular flow channel 47. The first diffuser cap opening 45 and the second diffuser cap opening 45a are disposed on opposite ends of the annular flow channel 47. Each of the first diffuser cap opening 45 and the second diffuser cap opening 45a are also in fluid communication with the opening of the suction lumen 42. Additionally, the diffuser cap openings 45 and 45a each have a normal vector 60 and 60a. The diffuser cap openings 45 and 45a can be disposed such that normal vectors 60 and 60a are parallel to tangent lines 62 and 62a, which are tangent to an outer circumference 15 of the transverse cross-section of the device taken along axis “T”.

Also, as illustrated in FIG. 5A, the first suction opening 45 can be oriented to face in a direction tangent to a first edge portion 63 of the outer surface of the outer wall of the tube. The second suction opening 45a can be oriented to face in a direction tangent to a second edge portion 63a of the outer surface 13 of the outer wall 12 of the tube 10. The first edge portion 63 and second edge portion 63a can be separated by a partial circumference 64 of the distal end portion of the elongate tube, which defines an annular flow channel 47 through which the first and second suction openings 45, 45a are disposed on opposite ends of said channel 47 each in fluid communication with the suction lumen.

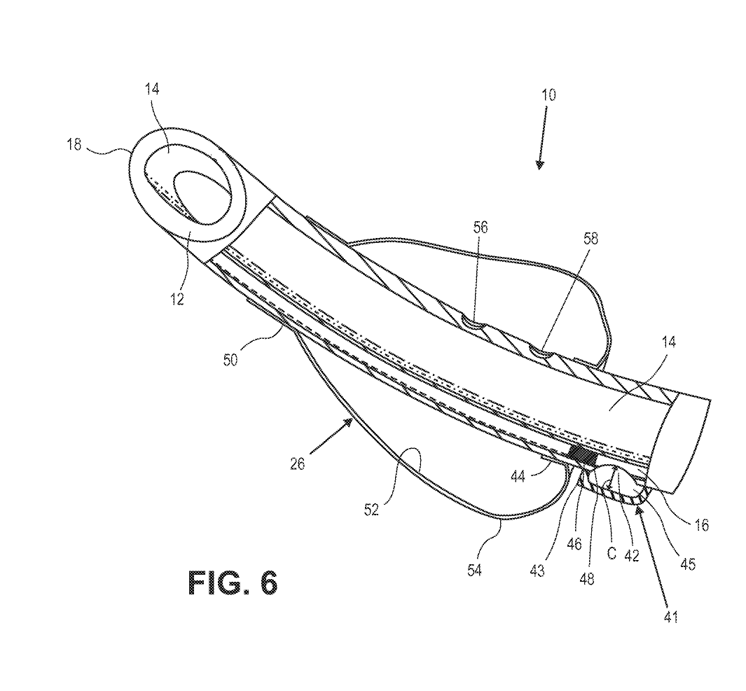

FIG. 6 illustrates a partially sectional view of the endotracheal tube and inflation lumen taken through axis ‘A,’ illustrated in FIG. 1. FIG. 6 shows the suction lumen 16 extending through the length of the endotracheal tube 10. The outer wall 12 of the endotracheal tube 10 defines an opening 42 in the suction lumen 16, just proximal of a proximal end 44 of the inflation cuff 26. A plug 46 can be positioned in the suction lumen 16 just distal to a distal end 48 of the opening 42. The plug 46 can be formed from UV glue or any other suitable material for sealing off the portion of the suction lumen 16 distal to the distal end 48 of the opening 42. In this way, the suction lumen 16 effectively terminates at the opening 42, where secretions accumulating around the proximal end of cuff 26 can be suctioned.

FIG. 6 also illustrates the inflation cuff 26. The cuff can include a distal end 50 and a proximal end 44, as well as an inner surface 52 and an outer surface 54. The inner surface 52 of the distal end 50 of the cuff 26 is bonded to the endotracheal tube 10 near its distal end 18. Preferably, the proximal end 44 of the cuff 26 can be folded in, or inverted, such that the outer surface 54 is bonded to the endotracheal tube 10. The bond can be formed by a suitable adhesive, melting, or any other form of bond that will connect the cuff 26 to the tube 10. Inverting the proximal end 44 of the cuff 26, but not the distal end 50, allows for the cuff 26 to increase in diameter from its distal end 50 to its proximal end 44. Additionally, the outer wall 12 of the endotracheal tube 10 defines openings 56 and 58 to allow fluid communication between the inflation lumen 28 and the inflation cuff 26.

FIG. 6 also illustrates the diffuser cap 41 positioned over the opening 42 to the suction lumen 16. The diffuser cap 41 includes an outer wall 43. The outer wall 43 defines diffuser cap openings 45 and 45a, which are in fluid communication with the opening 42 of the suction lumen 16. The diffuser cap 41 is raised above the outer surface 13 of the outer wall 12 of the endotracheal tube 10. The diffuser cap 41 can also be configured to span the entire circumference of the suction lumen opening 42, such that the entire opening 42 is enveloped by the diffuser cap 41.

FIG. 7 illustrates a diffuser cap for coupling to an endotracheal tube in accordance with an embodiment of the present invention. The diffuser cap 41 has an outer wall 43, which defines diffuser cap openings 45 and 45a. The diffuser cap 41 can be raised about the outer surface 13 of the outer wall of the endotracheal tube, by a distance of “C.” Distance “C” creates an annular flow channel 47 between the outer wall 43 of the diffuser cap 41 and the outer wall 12 of the endotracheal tube. Secretions and mucous can be suctioned into the flow channel 47. Additionally, the diffuser cap 41 can extend for a circumferential length “L.” Alternately, the diffuser cap 41 can extend for any length that is suitable for providing multi-port secretion suctioning for the endotracheal tube.

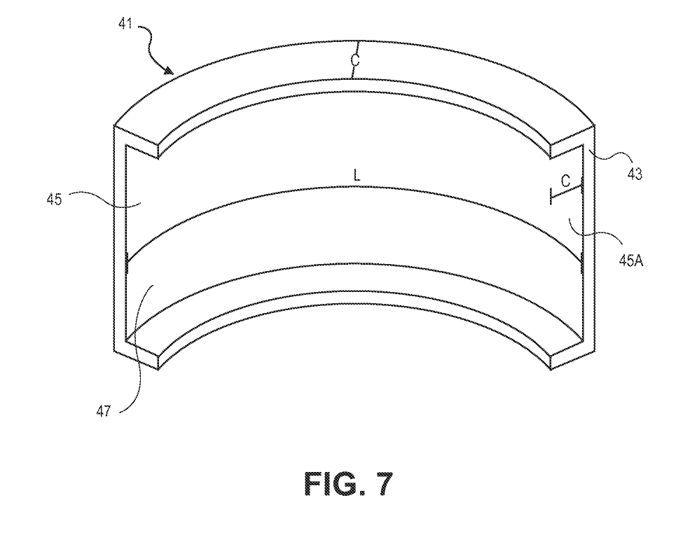

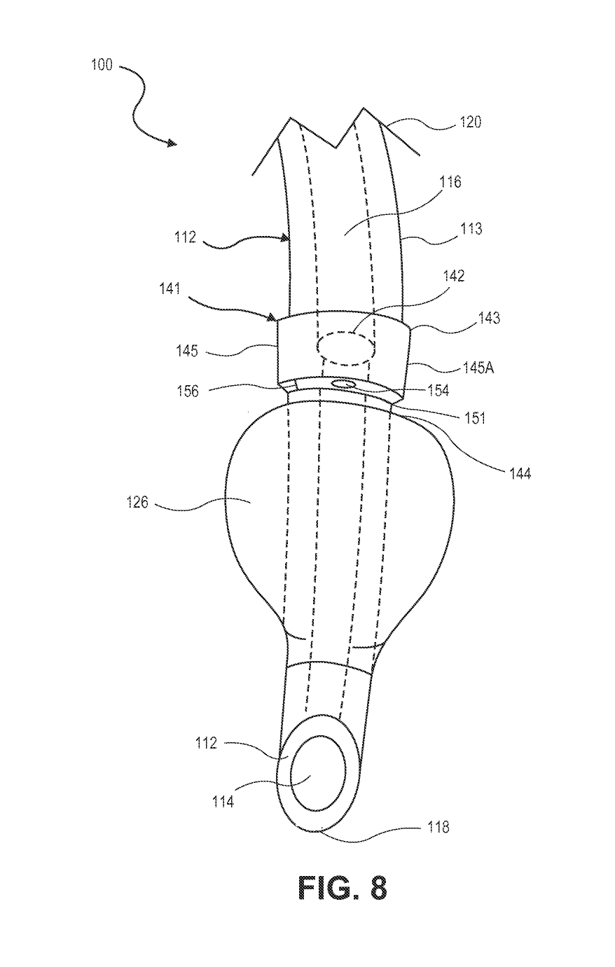

FIG. 8 illustrates an endotracheal tube having dual port subglottic suctioning in accordance with another embodiment of the present invention. As illustrated in FIG. 8, an endotracheal tube 100 includes an outer wall 112 which defines a generally cylindrical central lumen 114. The outer wall 112 also defines an elongate suction lumen 116. The endotracheal tube 100 has a longitudinal axis extending through the length of the tube 100 and one or more transverse axes “B” extending across the tube 100. The endotracheal tube has a distal end 118, which is inserted into the patient's trachea during the intubation procedure, and a proximal end 120, which is coupled to the respirator or other source of air for the patient. FIG. 8 also illustrates an inflation cuff 126 positioned proximate to the distal end 118 of the endotracheal tube 100. The inflation cuff 126 can be inflated via an inflation lumen defined by the outer wall 112 of the endotracheal tube 100.

Also, as illustrated in FIG. 8, a diffuser bridge 141 is positioned over an opening 142 defined by the outer wall 112 of the endotracheal tube 100. The opening 142 is in fluid communication with the suction lumen 116. The diffuser bridge 141 includes a diffuser bridge wall 143 which defines diffuser bridge openings 145 and 145a, which are in fluid communication with the opening 142 of the suction lumen 116 and the suction lumen 116.

As illustrated in FIG. 8, diffuser bridge openings 145 and 145a can be tangent to an outer surface 113 of the outer wall 112 of the endotracheal tube 100. The outer wall 143 of the diffuser bridge 141 can also define a third diffuser bridge opening 154 at a distal end of the diffuser bridge 141. The third diffuser bridge opening 154 can be positioned in the rise 156 of the outer wall 143 of the diffuser bridge which is perpendicular to the outer wall 112 of the endotracheal tube 100. The third diffuser bridge opening 154 can also be positioned parallel to a surface of a proximal end 144 of the inflatable cuff 126 in its inflated state. The third diffuser bridge opening 154 can provide additional suction to the area between the diffuser bridge 141 and the proximal end 144 of the inflation cuff 126. The diffuser bridge 141 can be configured to be raised slightly above the surface of the outer wall 112. However, the diffuser bridge 141 can also take any configuration suitable for providing fluid communication with the opening to the suction lumen 116 and the suction lumen 116.

Alternately, the diffuser bridge 141 of FIG. 8 includes two diffuser bridge openings 145 and 145a defined by the outer wall 143 of the diffuser bridge 141. The diffuser bridge openings 145, 145a are in fluid communication with the suction lumen opening 142 and the suction lumen 116, thus, creating a flow path for suctioned secretions into the suction lumen 116 and out of the endotracheal tube 100. The first diffuser bridge opening 45 can have a normal vector parallel to a line tangent to a left side of the outer circumference of the opening 142 for the suction lumen 116. The second diffuser bridge opening 145a can have a normal vector parallel to a line tangent to a right side 151 of the outer circumference of the opening 142 for the suction lumen 116.

In another embodiment of the diffuser bridge 141 shown in FIG. 8, the diffuser bridge 141 can be positioned on a distal portion 118 of the endotracheal tube 100. The diffuser bridge 141 can have the first diffuser bridge opening 145 having a normal vector parallel to a tangent to the outer circumference of a transverse cross section of the outer wall 112 of the endotracheal tube 100. The second diffuser bridge opening 145a can also have a normal vector parallel to a tangent to the outer circumference of a transverse cross section of the outer wall 112 of the endotracheal tube 100. The first diffuser bridge opening 145 can be oriented to face a direction that is tangent to a first edge portion of the outer wall 112 of the endotracheal tube 100.

In the embodiment of the diffuser bridge 141 shown in FIG. 8, the diffuser bridge 141 can extend around a partial circumference of the distal end portion of the elongate tube to define an annular flow channel. The first diffuser bridge opening 145 and the second diffuser bridge opening 145a are disposed on opposite ends of the annular flow channel. Each of the first diffuser can opening 145 and the second diffuser bridge opening 145a are also in fluid communication with the opening of the suction lumen 142.

The endotracheal tube 100 of FIG. 8 can also define the diffuser bridge 141. The endotracheal tube 100 can define the first diffuser bridge opening 145, such that it has a normal vector parallel to the tangent to the outer wall 112 of the endotracheal tube 100. The first diffuser bridge opening 145 can be in fluid communication with the suction lumen 116. The endotracheal tube 100 can also define the second diffuser bridge opening 145a, such that it has a normal vector parallel to the tangent to the outer wall 112 of the endotracheal tube 100. The second diffuser bridge opening 145a can be in fluid communication with the suction lumen 116. The first and second diffuser bridge openings 145 and 145a can also be oriented to be facing in a direction tangent to an edge portion of the outer wall 112 of the endotracheal tube 100. Alternately, the first and second diffuser bridge openings 145 and 145a can face parallel to respective lines tangent to respective portions of an outer circumference of the endotracheal tube 100. The distal end 118 of the endotracheal tube 100 can also define a partial circumference separating the first and second diffuser bridge openings 145 and 145a. The partial circumference defines an annular flow channel. The first and second diffuser bridge openings 145 and 145a are disposed on either end of this annular flow channel. The first and second diffuser bridge openings 145 and 145a are in fluid communication with the suction lumen 116, via the annular flow channel.

Also, as illustrated in FIG. 8, the diffuser bridge 141 can be disposed on top of the suction lumen opening 142. The diffuser bridge 141 can be raised above the outer surface 113 of the outer wall 112 of the endotracheal tube 100, by a distance of “C.” Distance “C” creates the annular flow channel between the outer wall 143 of the diffuser bridge 141 and the outer wall 112 of the endotracheal tube 100. Secretions and mucous can be suctioned into the flow channel and into the suction lumen 116 through suction lumen opening 142. Additionally, the diffuser bridge 141 can extend for a length “L,” which, as illustrated in FIG. 5, extends around a half of the generally cylindrical shape of the endotracheal tube 100. The diffuser bridge 141 can extend around a partial circumference of the distal end portion 118 Alternately, the diffuser bridge 141 can extend for any length that is suitable for providing multi-port secretion suctioning for the endotracheal tube.

Alternately, as illustrated in FIG. 8, the diffuser bridge 141 is positioned on a portion of the distal end 118 of the outer wall 112 of the endotracheal tube 100. The diffuser bridge 141 can be raised over the suction lumen opening 142 such that it at least partially covers the opening 142 to the suction lumen 116. A wall 143 of the diffuser bridge 141 and the endotracheal tube 100 can together define two suction openings 145 and 145a each facing parallel to respective lines tangent to respective portions of the outer circumference of the endotracheal tube 100. The diffuser bridge can also include the third diffuser bridge opening 154, which can be positioned facing the proximal end of the inflatable cuff in its inflated state.

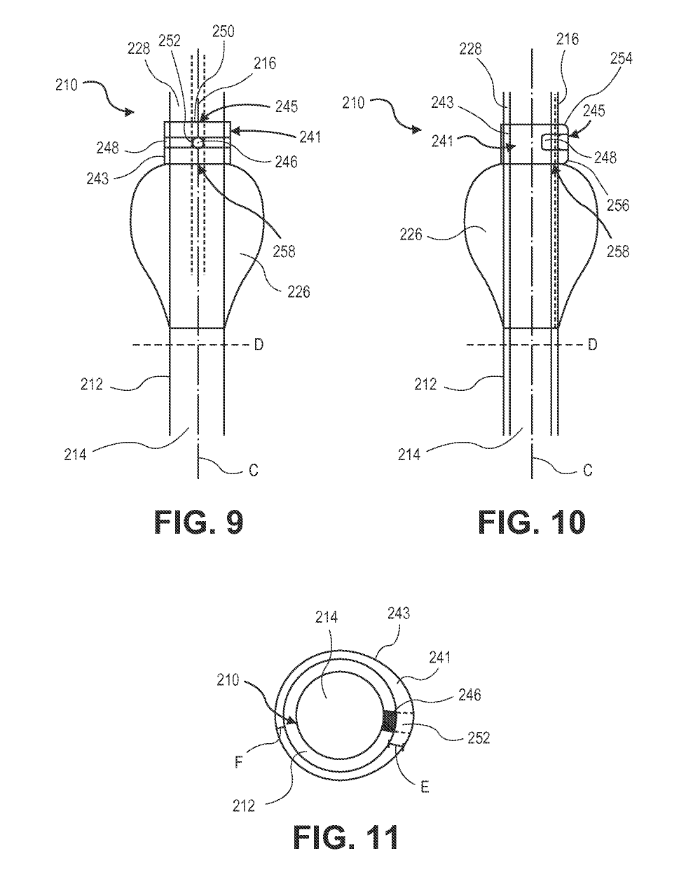

FIGS. 9 and 10 illustrate a partial view of an endotracheal tube having multi-port subglottic secretion suctioning in accordance with an embodiment of the invention. As illustrated in FIGS. 9 and 10 an endotracheal tube 210 in accordance with the invention includes an outer wall 212 which defines a generally cylindrical central lumen 214. The outer wall 212 also defines an elongate suction lumen 216. The endotracheal tube 210 has a longitudinal axis “C” extending through the length of the tube 210 and one or more transverse axes, such as axis “D” extending across the tube 210. FIGS. 9 and 10 also illustrates an inflation cuff 226. The inflation cuff 226 can be inflated via an inflation lumen 228 defined by the outer wall 212 of the endotracheal tube 210.

Also, as illustrated in FIGS. 9 and 10, a diffuser ring 241 is positioned around the endotracheal tube 210 at the point on the endotracheal tube 210 where a first suction opening 246 is defined by the outer wall 212 of the endotracheal tube 210 and positioned near a distal end of the endotracheal tube 210. The first suction opening 246 is in fluid communication with the suction lumen 216. The diffuser ring 241 includes a diffuser ring wall 243 which defines at least one diffuser ring channel 245, which is in fluid communication with the first suction opening 246 and the suction lumen 216.

As illustrated in FIGS. 9 and 10, the diffuser ring channel 245 can include a first diffuser ring slot 248 and a second diffuser ring slot 250. The first and second diffuser ring slot openings 248, 250 can be in fluid communication with the first suction opening 246. The first diffuser ring opening slot 248 can be parallel to the transverse axis “D” extending through the tube 210. The second diffuser ring opening slot 250 can be parallel to the longitudinal axis “C” extending along a length of the tube 210. Therefore, given this configuration, first diffuser ring opening slot 248 can be positioned perpendicular to second diffuser ring opening slot 250.

Also, as illustrated in FIGS. 9 and 10, a central ring opening 252 can be positioned at the intersection of the first and second diffuser ring opening slots 248, 250 and can be in direct fluid communication with the first suction opening 246. The diffuser ring 241 can be positioned around the endotracheal tube 210 such that the central ring opening 252 is directly over the first suction opening 246. Alternately, the diffuser ring 241 can be positioned in any way to facilitate suctioning of subglottic secretions into the suction lumen 216 of the endotracheal tube 210. Additionally, the edges of the diffuser ring 241 can be beveled to facitilitate subglottic secretion suctioning. Both the top edge 254 and the bottom edge 256 can be beveled. The edges of the first and second diffuser ring opening slots 248, 250 can also be beveled.

FIG. 11 illustrates a sectional view along axis “D” of the endotracheal tube 210 as illustrated in FIGS. 9 and 10. As illustrated in FIG. 11 the diffuser ring 241 can surround an outer surface of the outer wall 212 of the endotracheal tube 210. As noted with respect to FIGS. 9 and 10, the diffuser ring 241 can be positioned around the endotracheal tube 210 such that the central ring opening 252 is directly over the first suction opening 246. However, this positioning in the figures is not intended to be limiting. Alternately, the diffuser ring 241 can be positioned in any way that facilitates suctioning of subglottic secretions into the suction lumen 216 of the endotracheal tube 210. As illustrated in FIGS. 10 and 11, the diffuser ring 241 can be thicker over the first suction lumen opening 246. In other words, a first depth “E” of the diffuser ring is greater than a second depth “F” of the diffuser ring. Also as illustrated in FIGS. 9 and 11, the endotracheal tube can include a plug 258 positioned in the suction lumen 216 just distal to a distal end of the opening 246. The plug 258 can be formed from UV glue or any other suitable material for sealing off the portion of the suction lumen 216 distal to the distal end of the opening 246. In this way, the suction lumen 216 effectively terminates at the opening 246, where secretions accumulating around the proximal end of cuff 226 can be suctioned.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to falling within the scope of the invention.

1. An endotracheal tube assembly comprising:
an elongate tube having an outer wall defining an outer surface, the tube defining an elongate central lumen and an elongate suction lumen;
an inflatable cuff attached on a distal end portion of the elongate tube;
the elongate tube defining a first suction opening having a normal vector parallel to a tangent to an outer circumference of a transverse cross-section of the outer wall of the tube next to said first suction opening, the first suction opening being in fluid communication with the suction lumen.
